(19) United States
(12) Patent Application Publication
(10) Pub. No.: US 2024/0293099 A1
(43) Pub. Date: Sep. 5, 2024

(54) Sonographically-Aided Blood Vessel Penetration Device

(71) Applicants: Nicole Young, Somerville, NJ (US); Eric D. Young, Somerville, NJ (US)

(72) Inventors: Nicole Young, Somerville, NJ (US); Eric D. Young, Somerville, NJ (US)

(21) Appl. No.: 18/115,982

(22) Filed: Mar. 1, 2023

(51) Int. Cl. A61B 8/08; A61B 17/34; A61M 25/00

(52) U.S. Cl. CPC A61B 8/085; A61B 17/3403; A61M 25/0043; A61B 2017/3413

(57) ABSTRACT

A sonographically-aided blood vessel penetration device includes a casing defining an interior area. The penetration device includes a penetration assembly positioned in the interior area that defines a channel and may have a central venous catheter (CVC) that is slidable between a retracted configuration positioned entirely in the channel and a deployed configuration extending through the outlet and positioned partially outside of the interior area. A sonograph assembly is positioned on a bottom face of the casing adjacent the outlet for detecting a blood vessel. A digital display is positioned on a top face of the casing and in data communication with the sonograph module and is operative to display the detected blood vessel. An actuator assembly positioned on the top face of the casing is configured so as to move the CVC from the retracted configuration to the deployed configuration when actuated by a user.

### BACKGROUND OF THE INVENTION

This invention relates generally to blood vessel penetration devices and, more particularly, to a sonographically-aided blood vessel penetration device that uses Doppler or sonogram imaging to improve the accuracy of a medical technician in locating and piercing a blood vessel of vein of a patient.

Blood vessels, large veins, or radial arteries of a patient must be penetrated for the purpose of drawing blood or for administering medications or blood products. A catheter may be inserted into a main artery in the hand, neck, or groin with the intention of it remaining in in place for several days. Unfortunately, penetrating a suitable blood vessel structure with a large needle or catheter, especially of a patient who may be injured or dehydrated, is neither easy nor routine. Multiple penetrations of a patient's tissues are often required until a secure catheter/vein connection is made, each penetration seemingly more painful than the last.

One technology that is particularly effective at determining a position of a blood vessel and of a direction of blood flowing therein is ultrasonic Doppler sonography, also referred to as medical ultrasound. Medical ultrasound is a diagnostic technique used to create an image of internal body structures such as tendons, muscles, joints, blood vessels, and internal organs. Accordingly, it is feasible to use medical ultrasound technology to locate a blood vessel prior to attempting a penetration thereof.

Various devices have been proposed in the art for locating and more accurately penetrating a blood vessel. For instance, U.S. Pat. No. 5,080,103 discloses a syringe for Doppler sonographically aided penetration that uses Doppler sonography to indicate a blood vessel position. Specifically, the '103 syringe proposal includes a loudspeaker which either presents an audible indicator or actual audible sounds indicative of a blood flow direction. Even so, the located blood vessel and the parameters of its topography are not seen visually and missed penetration is still possible. Therefore, although presumably effective for their intended uses, there is still a need for a sonographically-aided device for locating and piercing a blood vessel that eliminates the physical gap between a Doppler receiver and a tip of a penetrating needle. In other words, there may be a seemingly unavoidable distance between an ultrasonic transceiver (i.e., transmitter and receiver combination) and a needle tip for penetrating the identified structure. The geometric relationship between an ultrasonic imaging or identification device and the penetration assembly is critically important.

Therefore, it would be desirable to have a sonographically-aided blood vessel penetration device that includes sonographic components that provide actual visualization of a blood vessel of a patient to be penetrated and a needle assembly that is immediately proximate and in-line with these sonographic components so as to minimize the risk of a missed penetration such as with a needle/catheter.

### SUMMARY OF THE INVENTION

A sonographically-aided blood vessel penetration device according to the present invention includes a casing having a rear wall and a continuous sidewall extending from a peripheral edge of the rear wall and a front wall opposite the rear wall, the rear wall, the sidewall, and the front wall, together, defining an interior area. The penetration device includes a penetration assembly positioned in the interior area that defines a channel and may have a central venous catheter (CVC) that is slidable between a retracted configuration positioned entirely in the channel and a deployed configuration extending through the outlet and positioned partially outside of the interior area. A sonograph assembly is positioned on a bottom face of the casing adjacent the outlet for detecting a blood vessel.

A digital display is positioned on a top face of the casing and in data communication with the sonograph module and operative to display the detected blood vessel. An actuator assembly positioned on the top face of the casing that is configured so as to move the CVC from the retracted configuration to the deployed configuration when actuated by a user.

Therefore, a general object of this invention is to provide a for identifying and penetrating veinous and arterial blood vessels.

Another object of this invention is to provide a sonographically-aided blood vessel penetration device, as aforesaid, having a needle that is slidably movable in-line with a pair of sonographic probes.

Still another object of this invention is to provide a sonographically-aided blood vessel penetration device, as aforesaid, that includes a visual display of structures beneath a patient's skin, e.g., the blood vessel to be penetrated.

Yet another object of this invention is to provide a sonographically-aided blood vessel penetration device, as aforesaid, in which a needle portion may be deployed so as to penetrate the very blood vessel being identified and modeled sonographically.

Other objects and advantages of the present invention will become apparent from the following description taken in connection with the accompanying drawings, wherein is set forth by way of illustration and example, embodiments of this invention.

### BRIEF DESCRIPTION OF THE DRAWINGS

FIG. 1a is a perspective view of a sonographically-aided blood vessel penetration device according to a preferred embodiment of the present invention;

FIG. 1b is a side view of the penetration device as in FIG. 1a;

FIG. 2a is a perspective view from a lower angle of the penetration device as in FIG. 1a, illustrated with a thermometer exploded from the casing and the needle in a retracted configuration;

FIG. 2b is an isolated view on an enlarged scale taken from FIG. 2a;

FIG. 3a is a front view of the penetration device has in FIG. 1b;

FIG. 3b is a sectional view taken along line 3b-3b of FIG. 3a, illustrating the penetration assembly at a retracted configuration;

FIG. 3c is an isolated view on an enlarged scale taken from FIG. 3b, illustrating a solenoid in a deployed or engaged configuration;

FIG. 4a is a front view of the penetration device has in FIG. 1b;

FIG. 4b is a sectional view taken along line 4b-4b of FIG. 4a, illustrating the penetration assembly at a deployed configuration;

FIG. 4c is an isolated view on an enlarged scale taken from FIG. 4b, illustrating a solenoid in a released configuration;

FIG. 5a is a side view of the penetration assembly according to a preferred embodiment of the present invention;

FIG. 5b is an exploded view of the penetration assembly as in FIG. 5a; and

FIG. 5c is an isolated view on an enlarged scale taken from FIG. 5b.

### DESCRIPTION OF THE PREFERRED EMBODIMENT

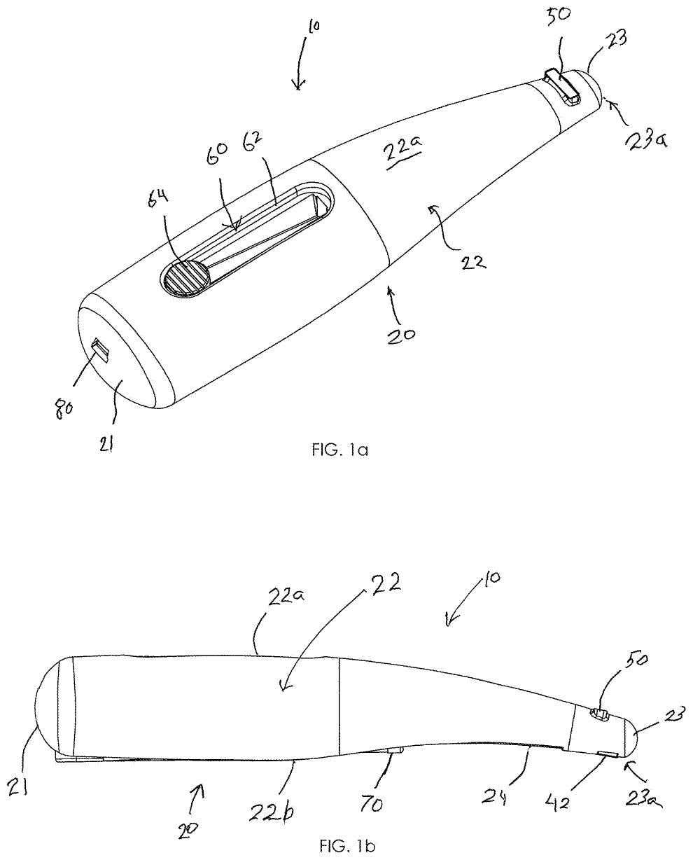

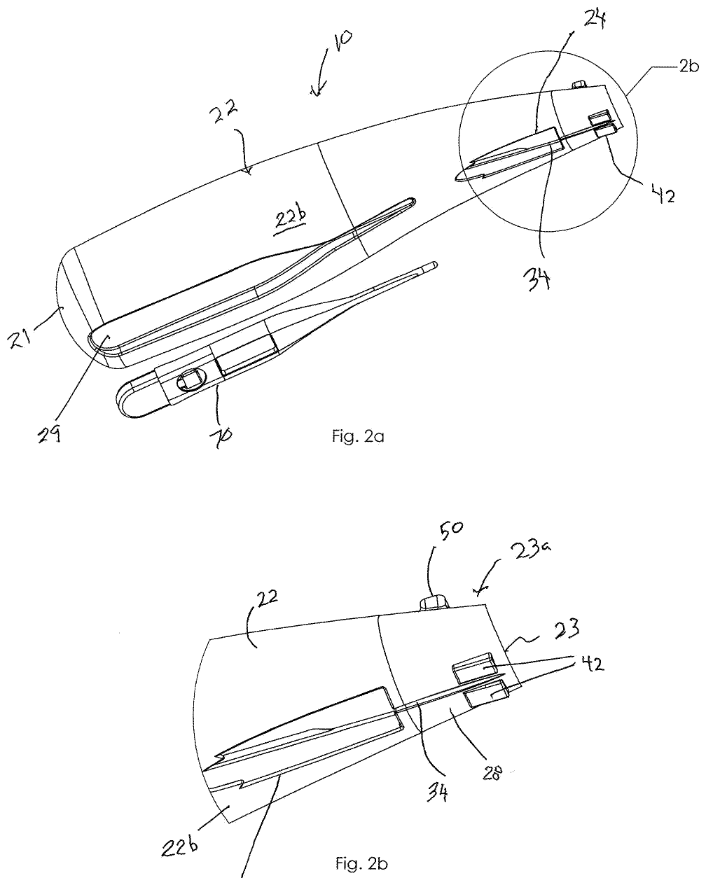

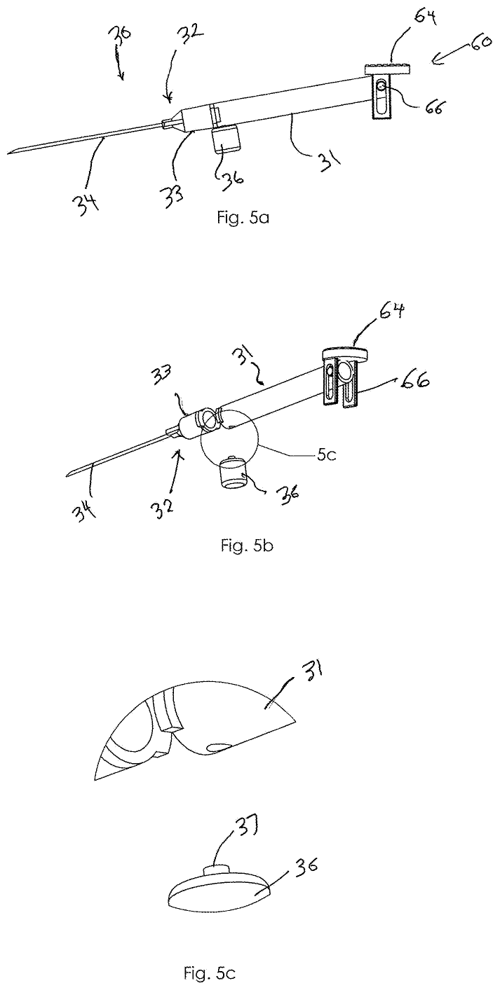

A sonographically-aided blood vessel penetration device according to a preferred embodiment of the present invention will now be described with reference to FIG. 1a to 5c of the accompanying drawings. The sonographically-aided blood vessel penetration device 10 is a medical device that may include a casing 20, a penetration assembly 30, a sonograph assembly 40, a digital display 50, and an actuator assembly 60.

The sonographically-aided blood vessel penetration device 10 includes a casing 20 having a rear wall 21, a continuous sidewall 22 that extends forwardly from peripheral edges of the rear wall 21 and a front wall 23 that is opposite the rear wall 21. Together, the rear, side, and front walls of the casing define an interior area in which several other components will be positioned as will be described later. Alternatively, the continuous sidewall 22 may be comprised of multiple interconnected side walls such that the casing 20 has a rectangular configuration or some other configuration. In addition, the rear wall 21 and front wall 23 may include outwardly rounded or bulbous configurations that are patient friendly and less likely to become entangled or snared in sheets, clothing, tubing, or other medical equipment. The continuous sidewall 22 includes a top face 22a and a bottom face 22b that will be referred to later with regard to the position of other components of the device. The bottom face 22b of the casing 20 defines an outlet 24 (i.e., a port or opening) adjacent the front wall 23 through which a needle may extend when a blood vessel has been identified and will be penetrated, as will be further described later. In addition, a portion of the interior area adjacent the front wall 23 may be referred to as a nose portion 23a configured to house electronics associated with the sonographic pads 42, digital display 50, or the like.

The penetration assembly 30 is substantially positioned within the interior area of the casing 20 and is configured to penetrate a blood vessel of a patient when actuated, such penetration as may be required when placing an intravenous or central line in a patient's arm, neck, chest, groin, or the like. A central venous catheter (CVC) 32 is most commonly inserted into a major vein to provide a central line to a patient's heart while a smaller needle may be inserted for general intravenous access. It is understood that all references to a CVC will be interpreted to include an intravenous insertion needle of any size or configuration. The penetration assembly 30 includes several components. More particularly, the penetration assembly 30 defines a channel 31 positioned within the interior area of the casing 20 and oriented at a downwardly angular configuration when viewing a downstream direction. The CVC 32 may include a hub 33 slidably positioned in the channel 31 and a needle 34 attached to the hub 33 and extending in a downstream direction away from the hub 33. In practice, the needle 34 and CVC 32 may be referred to as a syringe.

In a functionally related aspect, the sonographically-aided blood vessel penetration device 10 includes an actuator assembly 60 positioned on the top face 22a of the casing 20 and is operatively connected to the penetration assembly 30. In an embodiment, the actuator assembly 60 includes an actuation slider mechanism that defines a slot 62 along which in input member is selectively slidable by a user. Preferably, the input member is a slider button 64 that includes a surface grip configuration for receiving a user's finger to push the slider button 64 forwardly or rearwardly along the slot 62. A linkage 66, such as a lever or plurality of lever arms, may be coupled at one end to the slider button 64 and at another end to the hub 33 of the CVC 32. Accordingly, a slidable movement of the slider button 64 causes the hub 33 and CVC 32 to move downstream along the channel 31 toward the deployed configuration while a slidable movement of the slider button 64 and an upstream direction causes the hub 33 and CVC 32 to move upstream toward the retracted configuration. Stated another way, the hub 33 is slidably movable within the channel 31 between a retracted configuration positioned entirely in said channel 31 and a deployed configuration in which the needle 34 is extended through which the outlet 24 and positioned partially outside of the interior area.

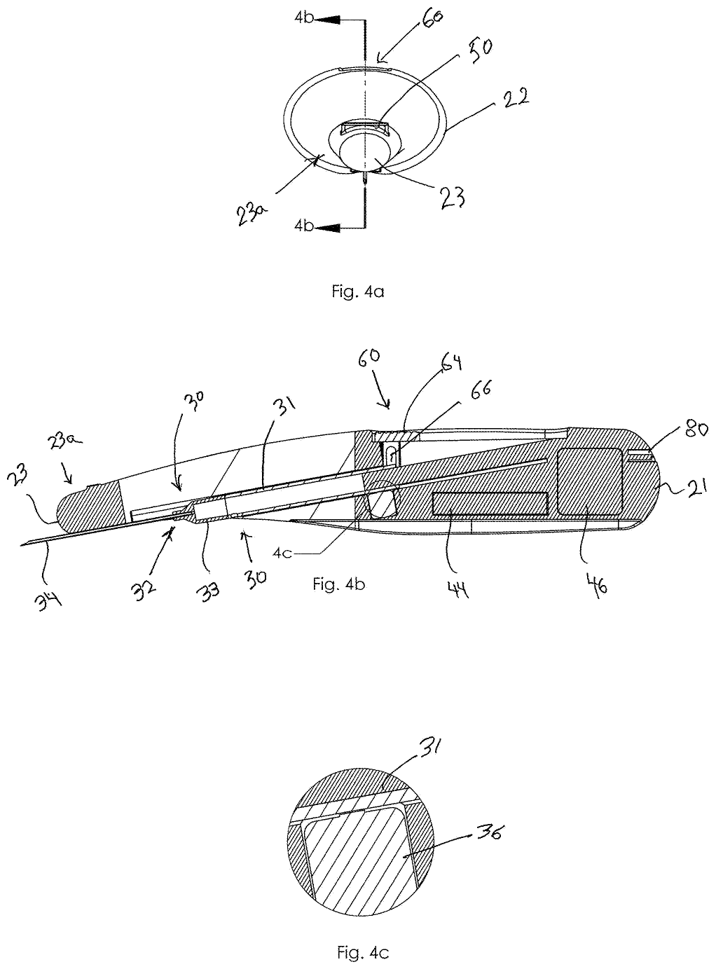

In a critical aspect, the sonographically-aided blood vessel penetration device 10 includes a sonograph assembly 40 for detecting the presence, position, and dimensions of a vein, or blood vessel, or the like in a patient and generating appropriate imaging data by which a digital display 50 may display the detected vein such that a technician or nurse may penetrate the detected vein by actuating the penetration assembly as described above. The digital display 50 may be in data communication with the sonogram module 44 such as with wires or may be wirelessly connected. More particularly, the sonograph assembly 40 may include at least one but preferably a pair of sensor pads 42 that may be coupled to the bottom face 22b of the casing 20, the pair of sensor pads 42 being in data communication with the electronics of a sonograph module 44 that may be positioned inside the interior area of the casing 20 or, in an embodiment, positioned remote from the casing 20 and in data communication via wireless signal transmission. The sensor pads 42 may include transmitters and receivers as would be known in the art. It is understood that the sonograph module 44 is capable of transmitting sound waves into a patient's body and receiving echoes in return and using this received data, creating an image such as, in the present invention, a picture of a blood vessel or vein that is being targeted for needle penetration. It is important that the pair of sensor pads 42 be mounted on the bottom face 22b of the casing 20 adjacent the outlet 24 so that the needle 34 is most likely to penetrate the detected vein when deployed as described above. The digital display 50, sensor pads 42, and deployed needle tip (when partially deployed) share a vertical (or near vertical) plane (FIG. 4b). It is understood that the sonograph assembly 40, including the sonograph module 44 and sensor pads 42, as well as the digital display 50 and any other electronic components may receive electrical current from a battery 46 That is also positioned in the interior area.

To further the accuracy of needle penetration, the bottom face 22b of the casing 20 defines a needle guide groove 28 that is in-line so as to extend through or in between the pair of sensor pads 42, the needle 34 being positioned to move slidably along or in this needle guide groove 28. To be clear, the positioning of the needle 34 so as to slide along the needle guide groove 28 through the sensor pads 42 of the sonograph assembly 40 and then through the outlet 24 is critical to the accuracy of needle penetration according to the present invention.

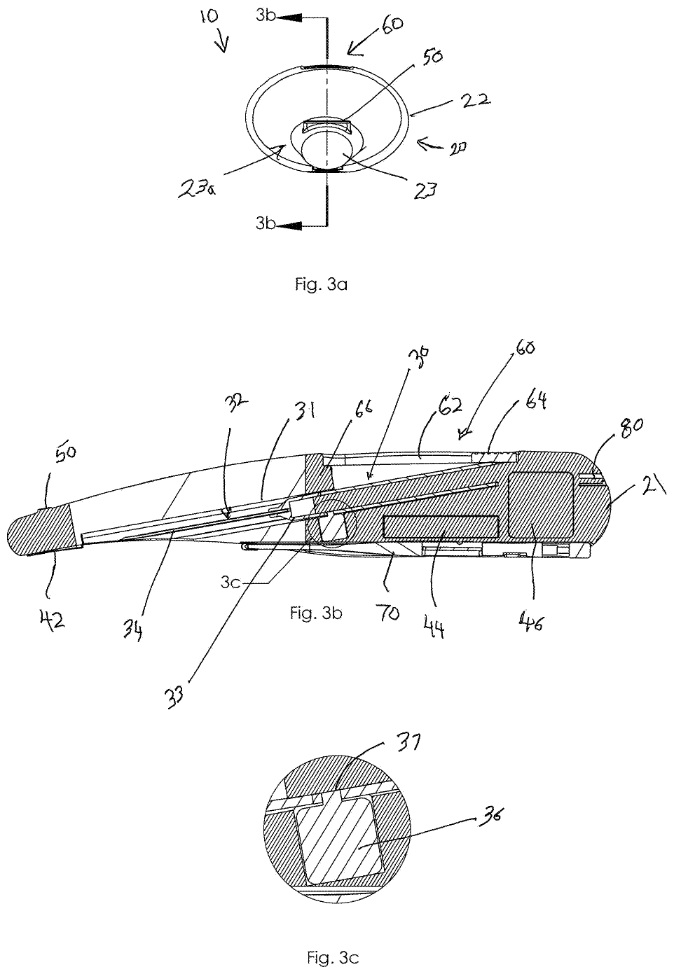

In another aspect, the sonographically-aided blood vessel penetration device 10 and, more particularly, the penetration assembly 30 includes a solenoid 36 or other fastener capable of selectively holding or releasing the CVC 32. More particularly, the solenoid 36 may be positioned proximate the downstream end of the channel 31 and may include a locking tab 37 that is movable between a 1) deployed configuration (also referred to as a locking configuration or an extended configuration) that extends through the channel and prevents the hub 33 associated with the CVC 32 from moving/sliding downstream in the channel 31 (FIG. 3b) and a 2) released configuration that allows the hub 33 associated with the CVC 32 to move slidably downstream in the channel 31 (FIG. 4b) and, in so doing, allowing the needle 34 to be deployed through the opening 24. In an embodiment, the solenoid 36 may be operatively coupled to and actuated by downstream movement of the slider button 64, by the linkage 66, or electronically (not shown).

In yet another aspect, the bottom face 22b of the casing 20 may define a sunken or recessed area 29 having a dimension that is large enough and having a shape configuration complementary to that of a medical grade digital thermometer 70. In an embodiment, the thermometer 70 may be selectively and frictionally held in the recessed area 29. Specifically, the recessed area 29 enables a hospital technician, e.g., a nurse, to store and transport a thermometer 70 for use in treating a patient and to avoid the thermometer 70 from becoming lost or unavailable when needed.

In yet another aspect, the sonographically-aided blood vessel penetration device 10 may include a USB port 80, such as along the rear wall 21. More particularly, a USB (an acronym meaning a universal serial bus) is a standard cable connection interface by which a plurality of peripheral electronic devices may be connected to an associated electronic device. A USB port enables an electronic device to be connected to external memory, external battery reserves, software input, or other peripheral devices. In the present invention, the USB port 80 enables the onboard battery 46 to be recharged and enables the computer software associated with the sonograph module 44 to be changed or updated over time.

In use, the front end of the sonographically-aided blood vessel penetration device 10 may be positioned with the bottom face 22b against a patient's skin proximate where a blood vessel or arterial vein is believed to be located even if it's exact outline is not apparent. The sonographic assembly 40 may be actuated to more specifically identify the vessel to be penetrated as described previously and a graphical representation thereof is displayed on the digital display 50. Once the blood vessel has been (and is currently being) visualized, the actuator assembly 60 may be manipulated so as to move the penetration assembly 32 to a deployed configuration in which a needle tip of the catheter or syringe penetrates the displayed blood vessel or vein.

It is understood that while certain forms of this invention have been illustrated and described, it is not limited thereto except insofar as such limitations are included in the following claims and allowable functional equivalents thereof.

1. A sonographically-aided blood vessel penetration device, comprising:
a casing having a rear wall and a continuous sidewall extending from a peripheral edge of said rear wall and a front wall opposite said rear wall, said rear wall, said sidewall, and said front wall, together, defining an interior area,
said casing having a bottom face that defines an outlet proximate said front wall;
a penetration assembly positioned in said interior area that defines a channel and has a central venous catheter (CVC) that is slidable between a retracted configuration positioned entirely in said channel and a deployed configuration extending through said outlet and positioned partially outside of said interior area;
a sonograph assembly having at least one sensor pad positioned on a bottom face of said casing and adjacent said outlet, said sonograph assembly being in data communication with an electronic sonograph module;
a display positioned on a top face of said casing and in data communication with said sonograph module;
an actuator assembly positioned on said casing that includes in input member proximate said top face and a linkage extending between said input member and said CVC, said linkage being slidable so as to cause said CVC to move from said retracted configuration to said deployed configuration when said input member is actuated by a user.

2. The blood vessel penetration device as in claim 1, wherein said actuator assembly is an actuation slider defining a slot along which said input member is selectively slidable so as to urge said CVC relative to said deployed or retracted configurations, respectively.

3. The blood vessel penetration device as in claim 2, wherein:
said input member is a slider button;
said linkage includes at least one lever coupled at one end to said slider button and coupled at another end to said CVC.

4. The blood vessel penetration device as in claim 1, wherein said bottom face of said casing defines a needle guide groove in-line with said at least one sensor pad, said CVC being positioned in said needle guide groove when said CVC is in said deployed configuration so as to penetrate a blood vessel identified by said sonograph module.

5. The blood vessel penetration device as in claim 1, wherein:
said at least one sensor pad includes a pair of sensor pads positioned adjacent one another and defining a space between said pair of sensor pads;
said casing defines a needle guide groove that is in-line with and extends through said space between said sensor pads; and
said CVC is positioned in said needle guide groove when said CVC is in said deployed configuration so as to penetrate a blood vessel identified by said sonograph module.

6. The blood vessel penetration device as in claim 4, wherein said channel is oriented in a downward angular configuration between a proximal end proximate said linkage and a distal end adjacent said at least one sensor pad.

7. The blood vessel penetration device as in claim 1, wherein said CVC includes a hub that is operatively coupled to said input member of said slider assembly and a needle coupled to and extending forwardly away from said hub.

8. The blood vessel penetration device as in claim 7, further comprising a solenoid positioned along said channel that is operative to move between an extended configuration that prevents movement of said hub in said channel and a released configuration that allows said hub to move in said channel according to downstream movement of said input member.

9. The blood vessel penetration device as in claim 7, further comprising a solenoid positioned along said channel that is operative to move between an extended configuration that holds said CVC at said retracted configuration and a released configuration that allows said CVC to move to said deployed configuration according to downstream movement of said input member.

10. The blood vessel penetration device as in claim 1 wherein:
said bottom face of said casing defines a recessed area; and
the blood vessel penetration device further comprising a thermometer removably seated in said recessed area.

11. A sonographically-aided blood vessel penetration device, comprising:
a casing having a rear wall and a continuous sidewall extending from a peripheral edge of said rear wall and a front wall opposite said rear wall, said rear wall, said sidewall, and said front wall, together, defining an interior area,
said casing having a bottom face that defines an outlet proximate said front wall;
a penetration assembly positioned in said interior area that defines a channel and has a central venous catheter (CVC) that is slidable between a retracted configuration positioned entirely in said channel and a deployed configuration extending through said outlet and positioned partially outside of said interior area;
a sonograph assembly having a pair of sensor pads positioned adjacent one another on said bottom face of said casing adjacent said outlet, said sonograph assembly being in data communication with an electronic sonograph module that is configured to detect a blood vessel;
a display positioned on a top face of said casing and in data communication with said sonograph module and operative to display said detected blood vessel;
an actuator assembly positioned on said top face of said casing that is configured so as to move said CVC from said retracted configuration to said deployed configuration when actuated by a user;
wherein:
said bottom face of said casing defines a recessed area; and
the blood vessel penetration device further comprising a thermometer removably seated in said recessed area.

12. The blood vessel penetration device as in claim 11, wherein said actuator assembly is an actuation slider that defines a slot and includes a slider button that is selectively slidable along said slot and which is operatively coupled via a linkage to said CVC so as to urge said CVC relative to said deployed or retracted configurations, respectively.

13. The blood vessel penetration device as in claim 11, wherein:
said bottom face of said casing defines a needle guide groove that is in-line with and extends between said pair of sensor pads; and
said CVC is positioned in said needle guide groove when said CVC is in said deployed configuration so as to penetrate a blood vessel identified by said sonograph module.

14. The blood vessel penetration device as in claim 12, wherein said channel is oriented in a downward angular configuration between a proximal end proximate said linkage and a distal end adjacent said pair of sensor pads.

15. The blood vessel penetration device as in claim 11, wherein said CVC includes a hub that is operatively coupled to said slider button of said slider assembly and a needle coupled to and extending forwardly away from said hub.

16. The blood vessel penetration device as in claim 15, further comprising a solenoid positioned along and adjacent to said channel that is operative to move between an extended configuration that prevents movement of said hub in said channel and a released configuration that allows said hub to move in said channel according to downstream movement of said input member.
